(19) United States
(12) Patent Application Publication
(10) Pub. No.: US 2002/0131933 A1
(43) Pub. Date: Sep. 19, 2002

(54) Biopolymer membrane and methods for its preparation

(76) Inventor: Yves Delmotte, Neufmaison (BE)

Correspondence Address: Janice Guthrie, Ph.D., BAXTER Healthcare Corporation, 17511 Armstrong Avenue, Irvine, CA, 92614, US

(21) Appl. No.: 10/004,257

(22) Filed: Oct. 26, 2001

Related U.S. Application Data
(63) Continuation-in-part of application No. 09/566,372, filed on May 8, 2000.
Application No. 09/566,372 is a continuation-in-part of application No. 09/386,198, filed on Aug. 31, 1999.
Application No. 09/386,198 is a continuation-in-part of application No. 08/679,658, filed on Jul. 12, 1996, now Pat. No. 5,989,215.
Application No. 08/679,658 is a continuation-in-part of application No. PCT/EP96/00160, filed on Jan. 16, 1996.

(51) Int. Cl. A61K 51/00; A61K 39/395; A61K 38/48; A61K 31/715; A61K 9/70

(52) U.S. Cl. 424/1.11; 424/443; 424/130.1; 424/94.64; 514/54; 514/2

(57) ABSTRACT

A biopolymermembrane in its substantially dry form having a thickness less than about 75 microns, a solvent content less than about 5% by weight of the membrane, a radius of curvature of less than about 5 centimeters, a density greater than about 1 g/cm³, and a maximum pore size of about 20 microns.

### CROSS-REFERENCE TO RELATED APPLICATIONS

This is a continuation-in-part application of U.S. Ser. No. 09/566,372 filed on May 8, 2000, which is a continuation-in-part U.S. Ser. No. 09/386,198 filed Aug. 31, 1999, which is a continuation-in-part of U.S. Ser. No. 08/679,658, filed on Jul. 12, 1996, now U.S. Pat. No. 5,989,215, which is a continuation-in-part of application no. PCT/EP96/00160, filed Jan. 16, 1995, all of which are incorporated herein by reference and made a part hereof.

### TECHNICAL FIELD

The invention relates to a biopolymer membrane having non-adhesive and anti-adhesion properties and that may be incorporated into a multilayered biopolymer structure that exhibits hemostatic, non-adhesive, and anti-adhesion properties.

### BACKGROUND OF THE INVENTION

One of the major problems in intra-abdominal surgery is the avoidance of post-operative adhesions. It is well known that adhesions contribute to pain, immobility, retarded wound healing, and in particular to intestinal obstruction, which may be life-threatening. In the field of gynaecological surgery, post-surgical adhesions involving female reproductive organs may result in infertility.

Each surgical procedure necessarily produces various forms of trauma where the abdominal cavity or other human cavity is opened for an inspection. Physiologically, the process of wound closure then starts when bleeding ceases upon formation of a haemostatic clot at the places where blood vessels are injured. The clot, at first comprising mainly platelets, is solidified by a fibrin network resulting from the activation of an enzyme cascade involving thrombin, factor XIII and calcium. Further steps on the way to the sealing of the wound are retraction of the haemostatic clot, invasion of various cell types including fibroblasts into the wound area and eventually the lysis of the fibrin network. Adhesions are thought to begin to form when the fibrin clot covering an injury comes into contact with a bleeding adjacent surface and the new connective tissue produced by the fibroblasts attach the two surfaces together.

The problems associated with adhesions often require a further operative procedure for removing/lysing the adhesions, called adhesiolysis, which, like the first operation, principally bears the risk of forming additional adhesions. Accordingly, the prevention of adhesion formation is medically important. Among the different approaches for prevention of adhesion formation is medically important. Among the different approaches for prevention of adhesion formation, one involves the use of materials as a physical or bio-mechanical barrier for the separation or isolation of traumatized tissues during the healing process. Both synthetic materials and natural materials have been used as a barrier to adhesion formation. Permanent, inert implants like Gore Tex® surgical membranes consisting of expanded polytetrafluoroethylene (PTFE) generally require a second operative procedure to remove them, while others such as surgical membranes of oxidized regenerated cellulose are biodegradable, but are thought to elicit an inflammatory response ultimately leading to adhesion formation (A. F. Haney and E. Doty, Fertility and Sterility, 60, 550-558, 1993).

Fibrin sealants and glues are well-known in the art for use in haemostasis, tissue sealing, and wound healing and have been commercially available for more than a decade. Use for anti-adhesion and drug delivery vehicle in glaucoma surgical procedures is one example. Fibrin glues mimic the last step of the coagulation cascade and are usually commercialized as kits comprising two main components. The first component is a solution comprising fibrinogen with or without factor XIII, while the second component is a thrombin calcium solution. After mixing of components, the fibrinogen is proteolytically cleaved by thrombin and thus converted into fibrin monomers. Factor XIII is also cleaved by thrombin into its activated form (FXIIIa). FXIIIa cross links the fibrin monomers to form a three-dimensional network commonly called “Fibrin Gel.”

As disclosed in the commonly assigned published PCT patent application, WO 96/22115, a self-supporting sheet-like material of cross-linked fibrin material can be used as a bio-mechanical barrier in the treatment of internal traumatic lesions, particularly for prevention of adhesion formation as a post-operative complication. The '115 Application discloses the mixing of a thrombin and calcium containing solution with a fibrinogen and Factor XIII containing solution. By using high thrombin concentrations to catalyze the conversion of fibrinogen into fibrin, the resulting fibrin material was found to be sufficiently rigid to be self-supporting and to have sufficiently small pore size to prevent the ingress of fibroblasts, which cause the formation of adhesions. The resulting fibrin material, however, did not readily retain water. In fact water could be easily expelled from the fibrin material by compressing the material by hand. Thus, this classic type fibrin material could not be used to deliver drugs to a wound site while being reabsorbed into the body during the fibrinolytic process.

U.S. patent application Ser. No. 09/566,019 filed on May 8, 2000, overcame these and other shortcomings in the prior art devices, by developing a fibrin material having a pore size less than about 5 microns. The small pore size helped define a void volume in the structure, whereby water could not escape from the structure. As such, the release of a drug incorporated into the water or buffer was regulated by passive diffusion, solubility, and the fibrinolytic process. Importantly, the '019 application discloses that the pore size in the fibrin clot is not necessarily directly related to the thrombin concentration when phosphate ions are present to react with the calcium in solution to prevent the collateral association of protofibrils.

U.S. patent application Ser. No. 09/566,372, which was filed on May 8, 2000, discloses that the a calcium inhibiting or blocking agent (such as those of the '019 application) can be used to produce a solid, porous fibrin structure having an open cross-section of more than 500 μm², which more closely resembles the porosity of natural human bone than the fibrin structures theretofore known. This particular structure is more useful as a bone glue or cement, as opposed to soaking up the exudates of an injury or preventing internal adhesions.

U.S. Pat. No. 5,989,215 discloses a fibrin sandwich formed in vivo by applying a single layer of fibrin glue to an injury site and then applying a second layer of a bio-mechanical fibrin barrier on top of the glue layer. The fibrin glue acts as a haemostatic agent and wound repair promoter, and the barrier layer is a self-supporting, sheet-like material of cross-linked fibrin that acts aids in the prevention of adhesions. The '215 patent further discloses that the thrombin concentration play a key function for controlling fibrin network formation. The '215 patent does not disclose a biopolymer structure having haemostatic, non-adhesive, and anti-adhesion properties where the structure can be formed in vitro. It also does not disclose that the fibrin network formation may be controlled by sequentially or simultaneously mixing the fibrinogen and thrombin that define the network.

The present invention is designed to solve these and other problems.

### SUMMARY OF THE INVENTION

The present invention relates to a biopolymer membrane having non-adhesive and anti-adhesion properties and that may be incorporated into a multilayered biopolymer structure with a biopolymer product where the structure exhibits hemostatic, non-adhesive, and anti-adhesion properties.

The biopolymer membrane of the invention exhibits non-adhesive and anti-adhesion properties. In one particular embodiment, the biopolymer membrane in its substantially dry form has a thickness equal to or less than about 75 microns a flexibility, as defined by a radius of curvature, of less than about 5 centimeters. The biopolymer membrane of this particular embodiment is further characterized by having a density greater than about 1 g/cm³, a maximum pore size in its hydrated form of about 20 microns, and a maximum pore size in its dehydrated form of about 10 microns.

The biopolymer membrane comprises a blend of a biomaterial, preferably fibrin or fibrinogen, and thrombin. The blend of the membrane may further comprise a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or any mixture thereof. In yet another embodiment, the biopolymer membrane further comprises an additive. In another embodiment, at least a portion of the biopolymer membrane is cross-linked. The membrane may also be sterilized.

In yet another embodiment, the present invention provides for a multilayered biopolymer membrane defined by a first biopolymer membrane and a second biopolymer membrane in contact with the first membrane, and wherein each membrane comprises a blend of a biomaterial, preferably fibrin or fibrinogen, and thrombin. The biomaterial selected for the first and second membranes may be the same or different. Each membrane may further comprise a second biomaterial.

The present invention also provides for a biopolymer product, preferably in contact with a biopolymer membrane to define a multilayered biocompatible structure. The biopolymer product also comprises a blend of a biomaterial, preferably fibrin or fibrinogen, and thrombin.

The blend of the biopolymer product may further comprise a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or a mixture thereof. In one particular embodiment, the biopolymer product has a water content of less than about 5% by weight.

In yet another embodiment, the biopolymer product may further comprise an additive selected from those disclosed as possible additives suitable for the biopolymer membrane. The biopolymer product may also be sterilized. In still yet another embodiment, the biopolymer product has at least one channel, which is formed by wrapping the biopolymer product around a support such as a mandrel. Each channel may contain a biomaterial, a living cell, a pharmaceutical agent, a therapeutic agent, or any material that does not degrade the biopolymer product. In another embodiment, the channel may be coated with a cross-linking agent. The biopolymer product may also contain an inner stent, an outer stent, or both. The biopolymer product may have any predetermined shape.

The biopolymer product may also be multilayered where at least two biopolymer products are in contact with each other. In another embodiment, any predetermined number of biopolymer product layers could then be formed to define a multilayered biopolymer product in contact with a biopolymer membrane, further defining a multilayered biocompatible structure of the invention. In this aspect of the invention, biopolymer product layers exhibit hemostatic properties, and the biopolymer membrane layers exhibit non-adhesive and anti-adhesion properties.

The present invention also provides a process for forming the biopolymer product and the biopolymer membrane. The initial step of the process comprises mixing, either sequentially or simultaneously, a biomaterial and thrombin in a solvent to define a gel. The biomaterial is selected from the group consisting of fibrin, fibrinogen, and blends thereof. In another embodiment, a second biomaterial may be added during the mixing step.

In one embodiment, the process further comprises the step of lyophilising the biomaterial before mixing it with the thrombin. Alternatively, the process comprises lyophilising both the biomaterial and the thrombin before mixing. In yet another embodiment, because the thrombin may be activable, the process further comprises the step of activating the thrombin, which may be done either before or after the mixing.

The next step in the process is drying the gel to define a biopolymer product having a solvent content from about 3% to about 35% by weight of the product. The process then comprises adjusting the solvent content of the biopolymer product so that the product is substantially filled with solvent. The final step of the process is compressing the biopolymer product to define a biopolymer membrane in its substantially dry form having a thickness equal to or less than about 75 microns, a solvent content less than about 5% by weight of the membrane, a radius of curvature of less than about 5 centimeters, a density greater than about 1 g/cm³, a maximum pore size its hydrated form of about 20 microns, and a maximum pore size in its dehydrated form of about 10 microns.

In yet another embodiment, the process further comprises adding an additive to the biopolymer membrane, to the biopolymer product, or both. In another embodiment, the process further comprises cross-linking at least a portion of the biopolymer membrane. The process also provides for sterilizing the biopolymer product, the biopolymer membrane, or both. In still yet another embodiment, the process further comprises forming at least one channel on a surface of the biopolymer product.

In another embodiment of the invention, an artificial skin is defined by contacting a biopolymer membrane to two sets of cells, where the first set of cells comprises a fibroblast, an endothelial cell, or a mixture thereof, and the second set of cells comprises an epithelial cell, a keratinocyte cell, or a mixture thereof.

Additional features and advantages of the present invention are described in, and will be apparent from, the drawings and the detailed description of the presently preferred embodiments.

### BRIEF DESCRIPTION OF THE DRAWINGS

FIGS. 1-3 show at different magnifications one embodiment of a dehydrated biopolymer membrane of the invention when fibrinogen and thrombin are simultaneously mixed.

FIGS. 4-6 show at different magnifications one embodiment of a hydrated biopolymer membrane of the invention when fibrinogen and thrombin are simultaneously mixed.

FIGS. 7-9 show at different magnifications one embodiment of a dehydrated biopolymer membrane of the invention when fibrinogen and thrombin are sequentially mixed.

FIGS. 10-12 show at different magnifications one embodiment of a hydrated biopolymer membrane of the invention when fibrinogen and thrombin are sequentially mixed.

FIG. 13 is a photograph of two biopolymer membranes of the invention being sutured together.

FIG. 14 is a schematic flowchart of the process of the invention.

### DETAILED DESCRIPTION OF THE PREFERRED EMBODIMENT

While this invention is susceptible of embodiment in many different forms, there is shown in the drawings, and will herein be described in detail, preferred embodiments of the invention with the understanding that the present disclosure is to be considered as an exemplification of the principles of the invention and is not intended to limit the broad aspect of the invention to the embodiments illustrated. The present invention provides for a multilayered biocompatible structure comprising a biopolymer membrane contacting a biopolymer product.

Biopolymer Membrane

The present invention provides for a biopolymer membrane that exhibits anti-adhesion properties. In one embodiment of the invention, the biopolymer membrane has a thickness equal to or less than about 75 microns and is characterized by a flexibility in its substantially dry form by a radius of curvature of less than about 5 centimeters, preferably less than about 3 centimeters, more preferably less than about 2.5 centimeters, and most preferably less than about 2 centimeters. What is meant by “substantially dry” is that the biopolymer membrane has a solvent content less than about 5% by weight of the membrane, preferably less than about 3%, and most preferably less than about 1%.

The biopolymer membrane of this particular embodiment is also characterized by a density greater than about 1 g/cm³, preferably greater than about 1.5 g/cm³, more preferably greater than about 1.6 g/cm³, even more preferably greater than about 1.7 g/cm³, and most preferably from about 1.75 g/cm³ to about 1.8 g/cm³. Finally, the biopolymer membrane of this embodiment has a maximum pore size in its hydrated form of about 20 microns, and a maximum pore size in its dehydrated form of about 10 microns, preferably about 5 microns, more preferably about 1 micron, even more preferably about 0.10 micron, and most preferably about 0.01 micron. What is meant by dehydrated is that more than about 50% of the solvent is removed from the membrane in its hydrated form. Naturally, and as seen in the drawings, the invention contemplates that the biopolymer membrane comprises a plurality of pores.

According to the invention, the biopolymer membrane comprises a blend of a biomaterial and thrombin. In one embodiment, the biomaterial is autologous. The biomaterial preferably comprises fibrin or fibrinogen. In another embodiment, the blend further comprises a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or any mixture thereof. One benefit of the second biomaterial is that it enhances the biopolymer membrane's ability to be used as a matrix for cell cultures.

In another embodiment, the biopolymer membrane may further comprise an additive such as processing aids (such as a cryoprotectant like glyercol, dimethyl sulfoxide or trehalose), a radioactive marker (such as Technitium-99-m-HDP or human serum albumin radiolabeled with an iodine isotope such as ¹³⁵I or ¹²⁵I), a calcium containing compound (such as hydroxyapatite, calcium phosphate, tricalcium phosphate, biphasic blends thereof, and the like), an antibody, an antimicrobial agent, an agent for improving the biocompatibility of the structure, proteins, an anticoagulant, an anti-inflammatory compound, a compound reducing graft rejection, any living cell (including but not limited to fibroblasts, chondrocytes, osteoblasts, stem cells), cell growth inhibitors, agents stimulating endothelial cells, antibiotics, antiseptics, analgesics, antineoplastics, polypeptides, protease inhibitors, vitamins, cytokine, cytotoxins, minerals, proteins, interferons, hormones, polysaccharides, genetic materials, proteins promoting or stimulating the growth and/or attachment of endothelial cells on the cross-linked biopolymer, growth factors, cell growth factors, growth factors for heparin bond, tannic acid, nerve growth factor, neurotrophic factor (NTFs), neurothrophin 3 (NT3), brain derived NTF (BDNTF), cilary NTF (CNTF), substances against cholesterol (such as statins or stanols, including but not limited to: Vastatin, Pravastatin, Simvastatin, Fluvastatin, Atorvastatin, and Cerivastatin), pain killers, collagen, osteoblasts, chondroblasts, chondrocytes, osteoclasts, hematpoeitic cells, stromal cells, material, triglycerides, fatty acids, C₁₂-C₂₄ fatty acids, collagen, any pharmaceutical agent (such as antibiotics, antiseptics, analgesics, antineoplastics, and the like), activable (preferably light activable) factor VII, activable (preferably light activable) factor IX, activable (preferably light activable) factor X, activable (preferably light activable) factor XI, activable plasmin, photoactivable t-PA, photoactivable urokinase, taxol, cytostatic agent, antigenic agent, plasminogen, compounds activating the conversion of plasminogen into plasmin (such as t-PA, u-PA, su-PA, streptokinase, alteplase, and the like), compounds inhibiting the conversion of plasminogen in plasmin (such as aprotinin, tranexanic acid, a2-antiplasmins, a2-macroglobulins, a2-antitrypsin, antithrombin, antistreptokinase, aminocapronic acid, tranexamic acid, Cl-esterase inhibitor, anti-urokinase, and the like), and mixtures thereof.

The present invention further provides that at least a portion of the biopolymer membrane may be cross-linked. The cross-linking can be effectuated with any cross-linking agent known in the art, preferably chosen so as not to substantially degrade the biopolymer membrane. What is meant by substantially in this instance is that the degradation occurs to a degree whereat the biopolymer membrane no longer exhibits any anti-adhesion properties. Preferable examples of a cross-linking agent include those selected from the group consisting of aldehydes, diimides, enzymes, tri-hydroxybenzene carboxylic acids, and mixtures thereof. A preferable tri-hydroxybenzene carboxylic acid is tannic acid. A preferable aldehyde is formaldehyde or glutaraldehyde. A preferable enzyme is factor XIII. Additionally, physical cross-linking methods may be used instead of the above chemical methods.

In yet another embodiment of the invention, the biopolymer membrane is sterilized. The sterilizing can be effectuated with any method known in the art, preferably chosen so as not to substantially degrade the biopolymer membrane. One purpose of sterilizing the membrane is to ensure the absence (to the best extent allowed by the method) of any agent, bacteria, virus, retrovirus (HIV), prions, or any composition that promotes an immunological response. What is meant by substantially in this instance is that the degradation occurs to a degree whereat the biopolymer membrane no longer exhibits any anti-adhesion properties. The sterilizing agent can be a physical agent such as heat, gamma beam radiation, ion-beam, electron-beam radiation, radio-frequency, and the like. Alternatively, the sterilizing agent can be a chemical agent such as ethylene oxide. The present invention contemplates that one sterilizing agent may be used in combination or in successive steps with another sterilizing agent. Further, the time duration of sterilizing the biopolymer membrane is not critical, provided that the purpose is met and the membrane is not degraded.

When heat sterilization is employed, the temperature is preferably from about 30° C. to about 150° C., more preferably from about 50° C. and about 125° C., and most preferably from about 80° to about 100° C. Alternatively, the membrane may be sterilized at a low temperature. A suitable low temperature is below 0° C., preferably below −20° C., more preferably below −40° C., and most preferably below −80° C. The low temperature sterilization can be carried out in combination with the use of a liquefied gas such as liquid nitrogen or the use of gaseous atmosphere at a low temperature as defined above.

The sterilization can also be carried out in a bath having one or more disinfecting agents. The bath may be aqueous, polar-organic, or a mixture thereof. The bath temperature is preferably between about 20° C. and about 150° C., and more preferably between about 50° C. and about 125° C., inclusive of the endpoints. When the temperature of the bath exceeds its boiling point, the sterilization can be carried out under pressure, such as in an autoclave.

The present invention also provides for a multilayered biopolymer membrane defined by a first biopolymer membrane and a second biopolymer membrane in contact with each other and wherein each membrane comprises a blend of a biomaterial and thrombin. The biomaterial selected for the first and second membranes may be the same or different. That is, like the biomaterial of the single layer membrane, the biomaterials for the first and second membranes preferably comprise fibrin, fibrinogen or a blend thereof. Each membrane may further comprise another biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or any mixture thereof. The two membranes are contacted with each other preferably by hydration and compression.

The inventors contemplate that the multilayered membrane may comprise any predetermined number of biopolymer membranes made according to the invention, where each layer (biopolymer membrane) of the multilayered membrane has a thickness equal to or less than about 75 microns; is characterized by a flexibility in its substantially dry form by a radius of curvature of less than about 5 centimeters, preferably less than about 3 centimeters, more preferably less than about 2.5 centimeters, and most preferably less than about 2 centimeters; has a solvent content less than about 5% by weight of the membrane, preferably less than about 3%, and most preferably less than about 1%; has a density greater than about 1 g/cm³, preferably greater than about 1.5 g/cm³, more preferably greater than about 1.6 g/cm³, even more preferably greater than about 1.7 g/cm³, and most preferably from about 1.75 g/cm³ to about 1.8 g/cm³; and has a maximum pore size in its hydrated form of about 20 microns, and a maximum pore size in its dehydrated form of about 10 microns, preferably about 5 microns, more preferably about 1 about micron, even more preferably about 0.10 micron, and most preferably about 0.01 micron.

The multilayered membrane will have a total thickness, which is the sum of all the its dehydrated form of about 10 microns, preferably about 5 microns, more preferably about 1 about micron, even more preferably about 0.10 micron, and most preferably about 0.01 micron.

The multilayered membrane will have a total thickness, which is the sum of all the biopolymer membranes comprising the multilayered membrane. The total thickness will depend on the user's need and can range from less than about fifty microns to any predetermined endpoint. A total thickness less than fifty microns is possible, but the membrane would need a support surface to be hydrated and positioned. Further, each layer of the membrane will have a respective density, which may be the same or different from the density of another layer. Like the total thickness, whether the layers have different or equivalent densities depends on the user's need. The densities of the respective layers of the biopolymer membrane can be controlled by varying the concentration of the thrombin, which will also vary the membrane's residence time such that a higher thrombin concentration results in an increased density and longer residence time.

The biopolymer membrane of the invention may be used as a physical barrier that covers tissues during wound-healing in order to avoid adhesions between tissue surfaces. The membrane may be employed in almost any clinical application such as gynecological surgery, myomectomy, metroplasty, conservative surgery for endometriosis, cardiac surgery, total artificial heart surgery, open heart surgery, banding and reconstruction of tissue deficiencies, patching blood vessels, general surgery, hernia repair, repair of large abdominal wall defect, tissue engineering, and the like. The biopolymer membrane of the invention may also be used for making heart socks, biochips, tablets, microparticles, granules, etc. The inventor contemplates that the membrane could be formed into any desired shape. The membranes of the invention may be employed in humans, and even bovines, horses, canines, felines.

Biopolymer Product

The present invention also provides for a biopolymer product. In a preferred embodiment, the biopolymer product contacts the biopolymer membrane to define a multilayered biocompatible structure. That is, the multilayered biocompatible structure of the invention may comprise one or more biopolymer membranes in contact with a biopolymer product. In fact, any predetermined number of biopolymer membranes may be contacted with any predetermined number of biopolymer products, in any order, to define a multilayered biocompatible structure. One advantage of the biopolymer product is its haemostatic properties. According a to a preferred embodiment, the biopolymer product is a sponge.

The biopolymer product comprises a second blend of a biomaterial and thrombin. Like the biomaterial of the membrane, the biomaterial of the product may be fibrin, fibrinogen, or a blend thereof. In one embodiment, the fibrin acts as glue between the biopolymer product and membrane to define the multilayered biocompatible structure. In another embodiment, the biomaterial may further comprise a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or a mixture thereof. Preferably, the biopolymer product comprises a blend of fibrinogen and thrombin.

The biopolymer product of the invention preferably has a low water content. What is meant by low in this instance is less than about 5% by weight of the biopolymer product is water, preferably of less than about 2%, and more preferably less than about 1% by weight. Additionally, the biopolymer product may further comprise an additive selected from those disclosed as possible additives suitable for the biopolymer membrane. The biopolymer product may also be, and is preferably, sterilized. The sterilizing can be effectuated with any method known in the art, including those applied to the biopolymer membrane.

In certain embodiments, the biopolymer product comprises at least one channel, preferably a plurality of channels, which is formed by wrapping the biopolymer product around a mandrel or other similar support. Perimeter edges of the biopolymer product may be glued together with fibrin glue, which is known in the art. The channel(s) is/are possibly filled with a porous or non-porous material such as a biomaterial (as defined above) or any living cell. Suitable living cells include fibroblasts, chondrocytes, osteoblasts, stem cells, and the like. The channel(s) may also be filled with a pharmaceutical agent, such as a drug, designed to induce a therapeutic effect and/or produce immunological response. In another embodiment, the channel may be coated with a cross-linking agent. In yet another embodiment, the wrapped membrane product can be provided with inner stent and/or an outer stent. According to a specific embodiment, the biopolymer product of the invention has a predetermined shape, such as a heart sock. Any predetermined shape is possible.

Like the biopolymer membrane, the biopolymer product may also be multilayered. The multilayered biopolymer product comprises at least two biopolymer products in contact with each other. When fibrin is present, it acts as a glue to contact one biopolymer product with another. The inventors contemplate that any predetermined number of biopolymer products may be contacted (by compression) with each other to form a multilayered biopolymer product, which would increase the product's absorption capacity and render it very useful for the absorption of blood during surgery. It is possible to form a biopolymer membrane layer and then form a biopolymer product on the surface of the biopolymer membrane. Any predetermined number of biopolymer product layers could then be formed to define a multilayered biopolymer product attached to a biopolymer membrane, further defining a multilayered biocompatible structure of the invention. Thus, the biopolymer product portion exhibits hemostatic properties, and the biopolymer membrane portion exhibits anti-adhesion properties.

Naturally, a multilayered biopolymer product comprises at least a first and a second biopolymer product in contact with each other. Each biopolymer product comprises a blend of a biomaterial with thrombin, wherein each biomaterial may be fibrin, fibrinogen, or a blend thereof. Each biopolymer product may further comprise a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, or a mixture thereof. Each biomaterial in the multilayered biopolymer product may be the same or of different chemical composition than another.

Process

The present invention also provides a process for forming the biopolymer product and the biopolymer membrane. The initial step of the process comprises mixing a biomaterial and thrombin in a solvent to define a gel. The mixing may be sequential or simultaneous. What is meant by sequential is that the biomaterial is applied to the thrombin, or vice versa. The sequential method differs from the simultaneous method in that air bubbles form within the biopolymer product that will remain after compression.

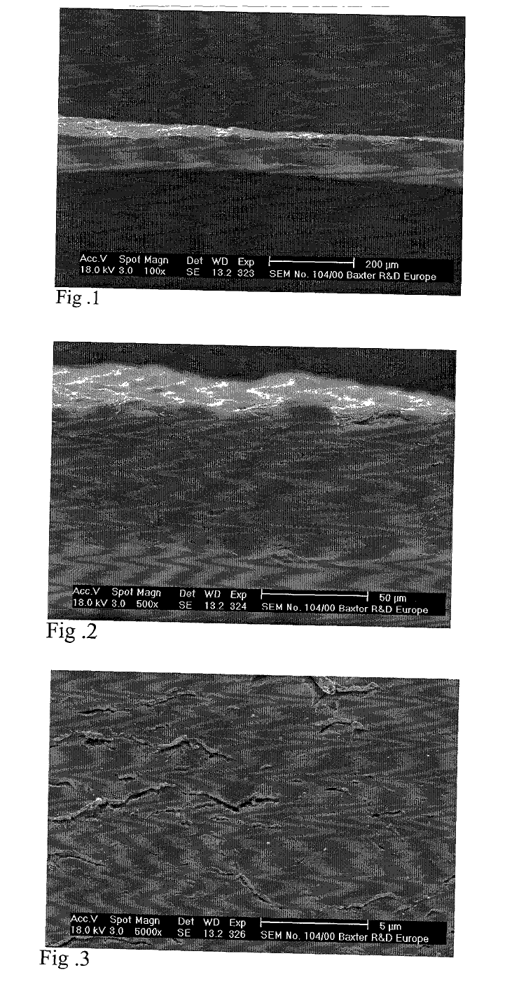

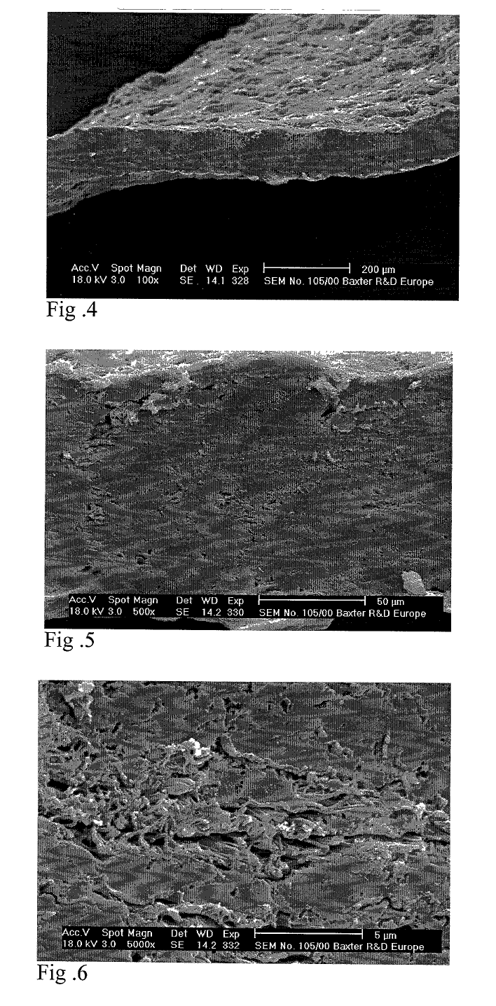

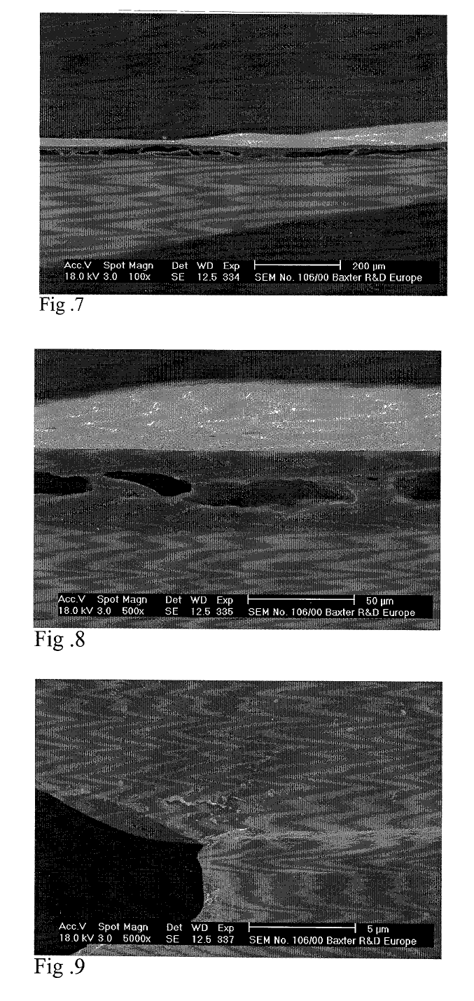

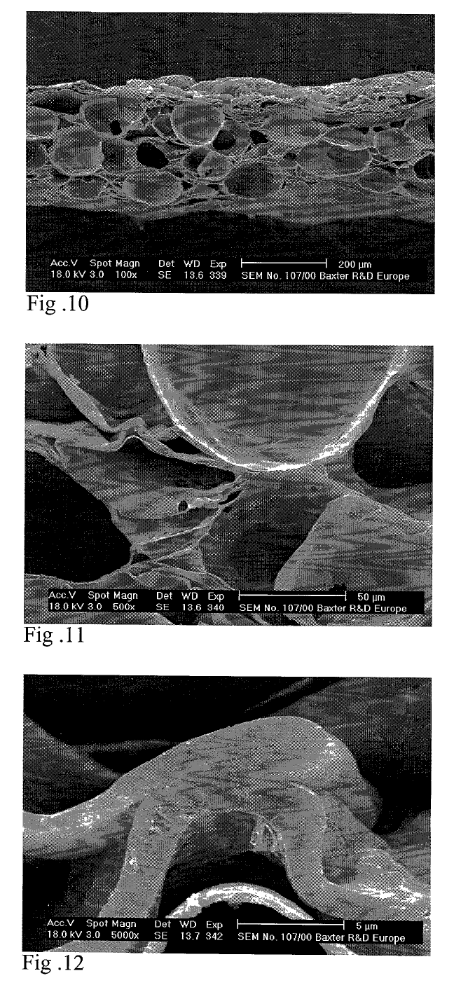

For example, FIGS. 1-3 show at different magnifications one embodiment of a dehydrated biopolymer membrane of the invention when fibrinogen and thrombin are simultaneously mixed. The average thickness of the dehydrated biopolymer membrane is about 70 to about 75 microns. FIGS. 4-6 show at different magnifications one embodiment of a hydrated biopolymer membrane of the invention when fibrinogen and thrombin are simultaneously mixed. The average thickness of the hydrated biopolymer membrane is about 115 to about 120 microns. FIGS. 7-9 show at different magnifications one embodiment of a dehydrated biopolymer membrane of the invention when fibrinogen and thrombin are sequentially mixed. The average thickness of the biopolymer membrane is about 40 to about 45 microns. FIGS. 10-12 show at different magnifications one embodiment of a hydrated biopolymer membrane of the invention when fibrinogen and thrombin are sequentially mixed. The average thickness of the biopolymer product is about 190 to about 200 microns.

As seen in Figures, the sequential mixing of the biomaterial and thrombin produces an alveolar structure, which is quite different than the membrane or product produced by simultaneously mixing the components. Upon hydration, the alveolar structure recovers part of its volume, resulting in the higher thickness ratio of the hydrated membrane to the dehydrated membrane in the biopolymer membrane produced by the sequential mixing of the biomaterial and thrombin versus the simultaneous mixing of the same components. Using the numbers from the figures, simultaneously mixing the biomaterial and thrombin produces a thickness ratio of about 1.6 to about 1.64 while sequentially mixing the biomaterial and thrombin produces a thickness ratio of about 4.44 to about 4.75.

Biopolymer membranes and products of desired thickness, porosity, and surface characteristics can be produced depending on the targeted surgical application. It is possible to produce one or more pores in the membrane having a diameter of less than about 0.10 micron, even less than about 0.01 micron. Alternatively, the alveolar structure could be obtained by introducing an inert gas stream.

The biomaterial is selected from the group consisting of fibrin, fibrinogen, and blends thereof. The biopolymer membrane may further comprise a second biomaterial such as chondroitin-4 sulfate, dermatan sulfate, keratan sulfate, hyaluronic acid, chitosan, chitin, alginate, laminin, elastin, fibronectin, collagen, proteoglycan, glycosaminoglycan, albumin, globulins, and mixtures thereof. Preferably, the biomaterial is essentially fibrin. What is meant by essentially is that if the biomaterial is a mixture, fibrin comprises at least more than about 40% by weight of the mixture. The second biomaterial may be the same, or is preferably of a different composition than, the first biomaterial.

In one embodiment, the biomaterial is lyophilized before being mixed with the thrombin.

In another embodiment, the biomaterial and the thrombin are both lyophilized prior to mixing, the gel being defined upon the addition of the solvent. It matters not in what sequence the solvent, the lyophilized biomaterial, and the lyophilized thrombin are combined for mixing. In a preferred embodiment, the biomaterial is fibrinogen, so that when it is mixed with thrombin, fibrin is formed. This embodiment is sometimes referred to as a “classic fibrin gel.” The thrombin or the biomaterial, or both, may be of natural origin or recombinant. In any embodiment, the thrombin may be activated or activable such as radiation-activable, photoactivable, and the like. If the thrombin is activable, the process comprises an additional step of activating the thrombin, which may be done either before or after the mixing.

According to one embodiment, the biomaterial is at least partly made from an autologous composition. One example of an autologous composition is a blood fraction of a patient to which the membrane will be applied, or of a body, preferably a human body, compatible with the patient to which the membrane will be applied. When using blood or one or more fractions of blood for the preparation of a membrane, the blood or fractions thereof are advantageously treated and/or sterilized to ensure the absence of any agent, bacteria, virus, retrovirus (HIV), prions, or any composition that promotes an immunological response.

The solvent of the process may be aqueous or organic, preferably chosen so as not to degrade substantially the biopolymer membrane. What is meant by substantially in this instance is that the degradation occurs to a degree whereat the biopolymer membrane no longer exhibits any anti-adhesion properties. Preferable organic solvents are polar-organic solvents, examples of which include but are not limited to cremophor and polyethyleneglycol. If PEG is used, it preferably has a molecular weight less than about 2,000, more preferably less than 1,000, and most preferably less than 600. The present invention contemplates the use of polyethyleneglycol derivatives, such as polysorbate 80, ester of polyethyleneglycol, and the like. Further, a mixture of solvents is contemplated, including a mixture comprising water and polar-organic solvent(s) or a mixture comprising polar-organic solvents.

The second step of the process is drying the gel to define a biopolymer product having a solvent content. Preferably, the biopolymer product is a sponge. The drying can be effectuated by any means known to those skilled in the art, including lyophilization, osmosis, centrifugation, compression, or a mixture thereof. The solvent content of the biopolymer product at this juncture is preferably from about 3% to about 35% by weight of the product, more preferably less than about 10%, even more preferably less than about 5%, and most preferably less than about 3%. Preferably, the biopolymer product has a water absorption capacity of about three times its own weight.

The third step of the process is adjusting the solvent content of the biopolymer product so that it is substantially filled with the solvent. What is meant by substantially in this instance is that the biopolymer product is preferably saturated with solvent. In a preferred embodiment, the solvent used in the adjusting step is substantially free, and most preferably completely free, of fibrinogen. The presence or absence of fibrinogen can be confirmed by SDS gel or capillary electrophoresis, or any method known in the art.

The final step comprising a process of the invention is compressing the biopolymer product to define a biopolymer membrane having a thickness equal to or less than about 75 microns and a solvent content less than about 5% by weight of the membrane, characterized in that the membrane has a radius of curvature of less than about 5 centimeters, a density greater than about 1 g/cm³, and a maximum pore size its hydrated form of about 20 microns, and a maximum pore size in its dehydrated form of about 10 microns, preferably about 5 microns, more preferably about 1 about micron, even more preferably about 0.10 micron, and most preferably about 0.01 micron.

Preferably, the biopolymer product is compressed at a pressure and during a time sufficient for reducing the amount of solvent in the gel to less than about 5% be weight of the biopolymer product, more preferably to less than about 2% by weight of the biopolymer product. In a preferred embodiment, the pressure exerted on the biopolymer product is greater than about 0.2 kg/cm², preferably greater than 0.5 kg/cm², more preferably greater than about 1 kg/cm², and most preferably greater than 2 kg/cm². In other embodiments, the exerted pressure is greater than about 10 kg/cm², preferably greater than about 50 kg/cm², and more preferably greater than 100 kg/cm², even more preferably greater than about 500 kg/cm², and most preferably less than or equal to about 5,000 kg/cm². The pressure used in the process will depend upon the chosen biomaterial(s) comprising the biopolymer product and the desired physical properties for the product. That is, the present invention contemplates any predetermined pressure, and those skilled in the art would employ a pressure applicable to desired purposes.

The compression of the biopolymer product is preferably carried out between at least two surfaces, one of which is at least partly porous for allowing the solvent in the biopolymer product to escape. In a preferred embodiment, the surfaces are porous so as to allow the solvent to escape on all sides. The present invention contemplates that the porosity of the surfaces may be the same or different. When surfaces of different porosities are employed, the biopolymer membrane comprises corresponding surfaces of different properties. For example, when using a substantially non-porous surface and a porous surface for the compression, the surface of the membrane will be relatively more dense, compact, and homogeneous at the point of compression with the non-porous surface than at the point of compression with the porous surface. Additionally, the porous portion of the compression surface may be operatively connected to a suction means such as a vacuum that aids in removing the solvent.

The compression step may comprise a series of at least two compressions, although any predetermined number of compressions is contemplated. When the compression is made in successive steps, with or without intermediate adjustments of the solvent content (such as rehydration), compression surfaces with varying (or the same) porosity may be used. For example, a first compression may be done with a compression surface or surfaces comprising pores that are larger in relative size to the pores of the compression surfaces of successive steps. For example, a first compression surface having a first pore size may compress the sponge to expel less than about 75%, preferably less than about 50%, of the solvent by weight of the sponge. Next, a second compression surface having a second pore size less than or equal to the first pore size compresses the biopolymer product. In one embodiment, the first compression surface has a first pore size larger than about 20 microns, and a second compression surface has a second pore size of less than or equal to about 20 microns, preferably less than about 10 microns. Naturally, the first and second compression surfaces may compress the sponge in a single step or in successive steps. Another aspect of the invention provides that the compression surface has a design such as an embossment, groove, protuberance, and the like. When such a compression surface is employed, the corresponding negative image of the design is transferred onto the biopolymer product during compression.

According to an embodiment of the process, before, after, or preferably during the compression step, the process further comprises stretching the biopolymer product, which serves to improve permeability and/or the porosity. The stretching may be in one, two, or three directions, which are preferably orthogonal. The stretching may be done by passing the biopolymer product between at least two rollers, one of which may be static. Alternatively, the stretching may be done between one roller and a support surface. One of the rollers may be static. If more than one rotating roller is used, the respective rotation speed of each the roller may be the same or different than the rotation speed of another roller. Different rotation speeds will result in different stretchings of the biopolymer product. Additionally, the biopolymer product may be compressed during the stretching step.

Further, if there is more than one compression step, the solvent content of the biopolymer membrane may be adjusted between some or all of the compression steps. The solvent used to adjust the solvent content of the biopolymer membrane may be the same or of a different composition than the solvent used to adjust the solvent content of the biopolymer product, but is preferably aqueous. This particular solvent may further comprise an additive such as an organic solvent (e.g., glycerol, polyethyleneglycol, and the like), processing aids, radioactive marker, calcium containing compounds, calcium phosphate, tricalcium phosphate, surfactants, lipids, fatty acids, betaines, fatty acid derivatives, disinfectants, virucides, methylene blue, bactericides, and the like. Preferably, the subsequent adjusting of the solvent content of the biopolymer membrane increases the solvent content before a successive compression step is executed. Even more preferably, the subsequent adjusting of the solvent content saturates the biopolymer membrane with solvent. The successive adjusting of the solvent content of the biopolymer membrane may be done by any means known within the art, including spraying the membrane with the solvent; dipping the membrane into the solvent; and/or pouring, dripping, or otherwise contacting the membrane and the solvent.

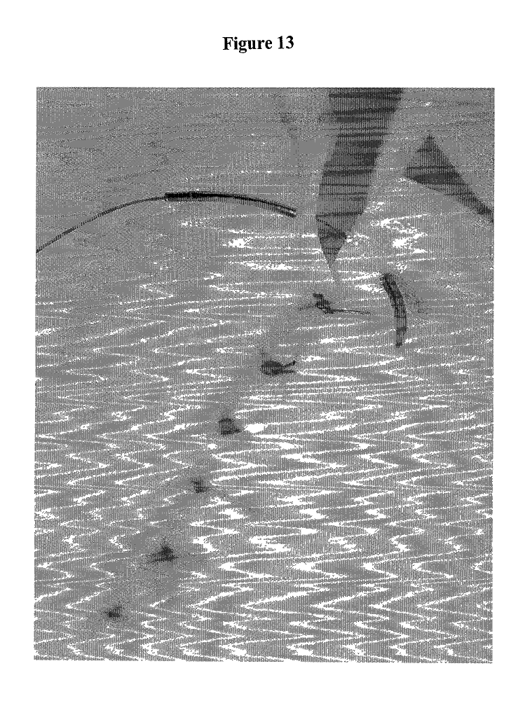

In another embodiment, at least one of the rollers comprises a cutting means such as knife, blade, scalpel, edge, scissors or the like. As shown in FIG. 13, the biopolymer membrane may be cut into two separate membranes and sutured together. Alternatively, two membranes of different compositions may be sutured together depending on the desired application. In yet another embodiment, a support surface such as a lattice may extend along a face of a biopolymer membrane, or may even be disposed inside the biopolymer membrane. A suitable lattice is oxidized methylcellulose, which is available commercially as INTERCEED™ from Johnson & Johnson.

In another embodiment, at least one roller has a controlled porosity. That is, the roller comprises a hollow cylinder having a substantially cylindrical surface with a plurality of apertures having a diameter from about 5 microns to about 500 microns, preferably from about 10 microns to 250 microns, and more preferably from about 50 microns to about 150 microns. In another embodiment, at least one roller further comprises a layer covering at least a portion of the roller. This layer comprises a plurality of apertures having a diameter from about 1 micron to about 20 microns, preferably from about 5 microns to 15 microns. It provides a second array of apertures to allow a fluid to flow through the membrane. The layer preferably comprises silicone, polytetrafluoroethylene, or a non-oxidizable metal such as inox.

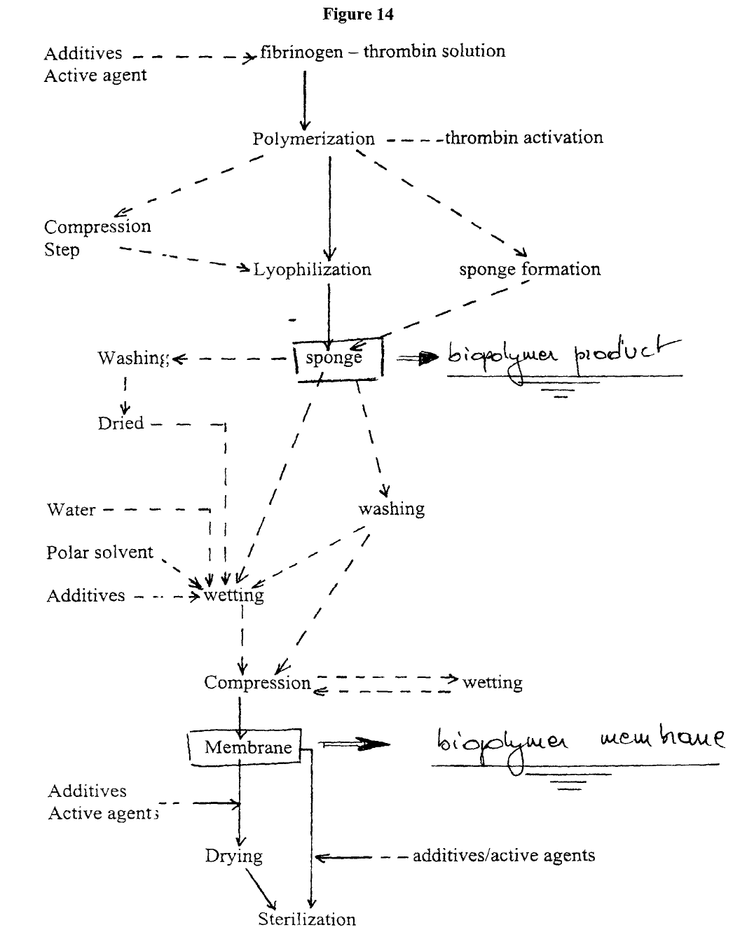

The process of the invention further provides for adding an additive to the biopolymer membrane and/or the biopolymer product. As shown in FIG. 14, the additive may be added at one or more points during the process. Suitable additives are processing aids (such as a cryoprotectant like glyercol, dimethyl sulfoxide or trehalose), a radioactive marker (such as Technitium-99-m-HDP or human serum albumin radiolabeled with an iodine isotope such as ¹³⁵I or ¹²⁵I), a calcium containing compound (such as hydroxyapatite, calcium phosphate, tricalcium phosphate, biphasic blends thereof, and the like), an antibody, an antimicrobial agent, an agent for improving the biocompatibility of the structure, proteins, an anticoagulant, an anti-inflammatory compound, a compound reducing graft rejection, any living cell (including but not limited to fibroblasts, chondrocytes, osteoblasts, stem cells), cell growth inhibitors, agents stimulating endothelial cells, antibiotics, antiseptics, analgesics, antineoplastics, polypeptides, protease inhibitors, vitamins, cytokine, cytotoxins, minerals, proteins, interferons, hormones, polysaccharides, genetic materials, proteins promoting or stimulating the growth and/or attachment of endothelial cells on the cross-linked biopolymer, growth factors, cell growth factors, growth factors for heparin bond, tannic acid, nerve growth factor, neurotrophic factor (NTFs), neurothrophin 3 (NT3), brain derived NTF (BDNTF), cilary NTF (CNTF), substances against cholesterol (such as statins or stanols, including but not limited to: Vastatin, Pravastatin, Simvastatin, Fluvastatin, Atorvastatin, and Cerivastatin), pain killers, collagen, osteoblasts, chondroblasts, chondrocytes, osteoclasts, hematpoeitic cells, stromal cells, osteoprogenitor cells, keratinocytes cells, anti coagulants, poly DL lactate, alginate, recombinant material, triglycerides, fatty acids, C₁₂-C₂₄ fatty acids, collagen, any pharmaceutical agent (such as antibiotics, antiseptics, analgesics, antineoplastics, and the like), activable (preferably light activable) factor VII, activable (preferably light activable) factor IX, activable (preferably light activable) factor X, activable (preferably light activable) factor XI, activable plasmin, photoactivable t-PA, photoactivable urokinase, taxol, cytostatic agent, antigenic agent, plasminogen, compounds activating the conversion of plasminogen into plasmin (such as t-PA, u-PA, su-PA, streptokinase, alteplase, and the like), compounds inhibiting the conversion of plasminogen in plasmin (such as aprotinin, tranexanic acid, a2-antiplasmins, a2-macroglobulins, a2-antitrypsin, antithrombin, antistreptokinase, aminocapronic acid, tranexamic acid, C1-esterase inhibitor, anti-urokinase, and the like), and mixtures thereof.

The process of the invention also provides for cross-linking at least a portion of the biopolymer membrane. The cross-linking can be effectuated with any cross-linking agent known in the art, preferably chosen so as not to substantially degrade the biopolymer membrane. What is meant by substantially in this instance is that the degradation occurs to a degree where at the biopolymer membrane no longer exhibits any anti-adhesion properties. Preferably examples of a cross-linking agent include those selected from the group consisting of aldehydes, diimides, enzymes, tri-hydroxybenzene carboxylic acids, and mixtures thereof. A preferable tri-hydroxybenzene carboxylic acid is tannic acid. A preferable aldehyde is formaldehyde or glutaraldehyde. A preferable enzyme is factor XIII.

The process of the invention further provides for sterilizing the biopolymer product and/or the biopolymer membrane. The sterilizing can be effectuated with any method known in the art, and can be performed for any predetermined duration, preferably chosen so as not to substantially degrade the biopolymer product or membrane. One purpose of sterilizing the biopolymer product and/or membrane is to ensure the absence (to the best extent allowed by the method) of any agent, bacteria, virus, retrovirus (HIV), prions, or any composition that promotes an immunological response. What is meant by substantially in this instance is that the degradation occurs to a degree whereat the biopolymer product no longer exhibits its hemostatic properties or the biopolymer membrane no longer exhibits its anti-adhesion properties. The sterilizing agent can be a physical agent such as heat, gamma beam radiation, ion-beam, electron beam radiation, and radio-frequency. Alternatively, the sterilizing agent can be a chemical agent such as ethylene oxide. The present invention contemplates that one sterilizing agent may be used in combination or in successive steps with another sterilizing agent.

When heat sterilization is employed, the temperature is preferably from about 30° C. to about 150° C., more preferably from about 50° C. and about 125° C., and most preferably from about 80° to about 100° C. Alternatively, the membrane may be sterilized at a low temperature. A suitable low temperature is preferably below 0° C., more preferably below −20° C., and most preferably below −40° C. The low temperature sterilization can be carried out in combination with the use of a liquefied gas such as liquid nitrogen or the use of gaseous atmosphere at low temperature.

The sterilization can also be carried out in a bath further comprising one or more disinfecting agents. The bath may be aqueous, polar-organic, or a mixture thereof. The bath temperature is preferably between about 20° C. and about 150° C., and more preferably between about 50° C. and about 125° C., inclusive of the endpoints. When the temperature of the bath exceeds its boiling point, the sterilization can be carried out under pressure, such as in an autoclave. In a preferred embodiment, the sterilization step of the process is performed after the washing the biopolymer product and/or membrane.

The washing step may be a single step or a series of successive steps and may be carried out in any solvent known in the art, provided that the biopolymer product and/or membrane is not substantially degraded. In another embodiment, the sterilizing step is carried out after washing the biopolymer membrane, and after drying the biopolymer membrane so to reduce the solvent content to less than about 1% by weight of the membrane, preferably less than about 0.5%, more preferably less than about 0.2%, and most preferably to less than about 0.1% by weight.

The residence time and/or biodegradability of the membrane can be shortened or lengthened by those skilled in the art by adapting certain experimental parameters to the desired need. For example, when fibrin is the biopolymer of choice, the residence time can be changed by adapting one or more of the following parameters: the thrombin and/or fibrinogen concentration; the presence or absence calcium-complexing agents (such as phosphate buffer solution, sodium citrate, EDTA, EGTA, 5,5′Br₂-BAPTA, 5N-BAPTA, ethyleneglycol-bis-(beta-aminoethylether)-N,N,N′,N′-tetraacetic acid, and the like); the presence or absence of plasminogen; the presence of a compound that activates the conversion of plasminogen into plasmin, such as t-PA, u-KA, su-PA, streptokinase and the like (the presence of which increases bio-degradability or bio-resorbability); the presence of compound that inhibits the conversion of plasminogen in plasmin, such as aprotinin, tranexanic acid, a2-antiplasmins, a2-macroglobulins, a2-antitrypsin, antithrombin, antistreptokinase, aminocapronic acid, tranexamic acid, C1-esterase Inhibitor, anti-urokinase, and the like (the presence of which decreases the biodegradability or the bio-resorbability); the concentration of the cross-linking agent (higher cross-linking results in decreased biodegradability) the compression; the degrees of porosity and the size of the pores; the thickness of the membrane; and the density of the membrane.

The process of the invention further provides for forming a channel, preferably a plurality of channels, on a surface of the biopolymer product. A channel is formed by placing the biopolymer product in a mold, preferably applying pressure of sufficient magnitude and time to the mold and/or to the biopolymer product, in order to form the desired channel. One particular embodiment provides the mold with at least one recess or other opening to allow for the escape of solvent. The channel may also be formed by inserting a mandrel, a tube, or other support into the biopolymer product, again preferably applying pressure of sufficient magnitude and time to the mandrel or other support and/or to the biopolymer product. In one embodiment, the polymerization of the biomaterial and thrombin occurs around the support, thus shaping the resulting biopolymer product.

Artificial Skin and Kit Therefor

The present invention further provides for an artificial skin comprising a biopolymer membrane, a first set of cells contacting the biopolymer membrane, and a second set of cells contacting the biopolymer membrane. What is meant by contacting is that the first and second set of cells may be mixed into, blended with, dissolved in, suspended in, or otherwise associated with the biopolymer membrane of the invention. In one particular embodiment, the artificial skin may be prepared from a kit, as is set forth below.

According to one embodiment, the first set of cells is a fibroblast, an endothelial cell, or a mixture thereof. The second set of cells is an epithelial cell, a keratinocyte cell, or a mixture thereof. Preferably, both sets of cells are biocompatible and do not produce an immunological response. The particular amount of each set of cells will depend on the desired use and can be determined by those of ordinary skill in the art. For example, if the artificial skin were to be used in a human body, the ratio of the first and second sets of cells per unit of measured centimeter in a hydrated biopolymer membrane (i.e., per square centimeter) would be nearly equal to that ratio present number in the same unit of measure in a human, preferably the particular human for which the artificial skin is targeted for use.

The first component of the kit is to prepare a biopolymer product comprising fibrin and place it in a first bag. The first bag housing the biopolymer product is then sterilized with gamma rays at about 25 KGray. A second bag comprising fibroblast cells is prepared, while a third bag comprising epithelial and/or keratinocyte cells is prepared. The second and third bags are connected to the first bag with sterile conduits.

The fibroblast cells of the second bag are introduced into the first bag through a conduit, hydrating the biopolymer product with cultured dermal fibroblasts. Possibly, the fibroblasts are further cultured on the biopolymer product. The temperature of the biopolymer product temperature of the sponge is then increased to or above about 0° C., preferably to or above about 4° C., more preferably to or above about 10° C. but less than or equal to about 37° C. Next, the first bag is compressed such that a biopolymer membrane is formed. During the compression, solvent from the first bag is expelled into the second bag through the conduit, or possibly through another opening or aperture of the first bag. The compression can be carried out by applying a vacuum on the second bag or by compressing the first bag between two plates. The compression is carried out so as to keep fibroblast cells in the inner portion of the membrane, so as to not damage the fibroblast cells.

The third bag having epithelial and/or keratinocyte cells is then conducted on the membrane in order to seed the membrane with the cells in the third bag, thus defining the artificial skin of the invention. The artificial skin of the invention can be stored at a low temperature, preferably lower than about −20° C., more preferably lower than about −50° C., and most preferably lower than about −80° C.

The endothelial cells of this embodiment may be isolated from the blood of a human or animal patient or from a biocompatible blood (for example from large vessel such as vein and/or microvascular sources such as fat). Preferably, the endothelial cells are autologous. One suitable example of a source of endothelial cells is abdominal wall fat that is removed from the patient by liposuction. Such cells are preferably concentrated more than about 1,000,000 cells per gram of fat, thus obviating the need to culture the cells. Not with standing, the cells may be cultured. Preferably, the cells are treated before being combined with the biopolymer product. Suitable treatment includes collagenase digestion and successive centrifugations with washing the cells between centrifugations. One possible procedure is centrifuging the cells at about 100 G for 4 minutes, discarding the supernatant, washing the cells, centrifuging again at about 100 G for about 3 minutes, and discarding the supernatant. The so-washed cells are then introduced into the third bag. Each bag of the kit is a cell culture bag and preferably comprises a gas-permeable (e.g., CO₂/O₂) polymer such as polyvinylchloride, polyethylene, polystyrene and the like. Each bag also preferably has means to allow the passage of such gases. Suitable means include tubing, valves, conduits, or other apertures.

In another embodiment, an artificial skin is prepared from a plasma, preferably an autologous or biocompatible plasma. The plasma is then mixed with fibroblast and/or endothelial cells to define a mixture. Thereafter, thrombin is added to the mixture to define a gel. The gel is then compressed to define a membrane having fibroblast and/or endothelial cells. The membrane can then be seeded with epithelial and/or keratinocyte cells. If such seeding is performed, the temperature of the membrane should be adjusted to about 0C., more preferably to or above about 4° C., most preferably to or above about 10° C. but less than or equal to about 37° C.

One formed, the membrane may be stored at low a temperature, preferably lower than about −20° C., more preferably less than about −50° C., most preferably less than about −80° C. Before using the membrane, though, its temperature is preferably increased to or above about 0° C., more preferably to or above about 4° C., most preferably to or above about 10° C. but less than or equal to about 37° C. Also before use, the endothelial or fibroblast cells present in the membrane and/or the epithelial and/or keratinocyte cell present on the surface of the membrane are preferably cultured for an effective amount of time to ensure a sufficient cell population such that confluent growth occurs.

As with other examples described herein, the thrombin may be activable, such as a photoactivable. The plasma-thrombin mixture can be stored in a dark environment such that the thrombin is not activated. Such an environment may comprise a bag as described above further having a light-protecting layer such that the thrombin does not activate. The plasma may also be premixed with endothelial and/or fibroblast cells. Once the thrombin is activated, a gel of the invention is formed, which can then be compressed and then seeded with epithelial and/or keratinocyte cells.

### EXAMPLES

### Example 1

Twenty milliliters at 80 mg/mL of Tissucol® (commercially available from IMMUNO AG) was dissolved in an aqueous aprotinin solution having 3000 KIU/ml and then diluted at a 1:4 ratio with distilled water to yield a fibrinogen concentration of approximately 20 mg/ml. Naturally, a higher concentration of fibrinogen may be used to yield a more dense and compact membrane. Next, approximately 20 milliliters of this solution was rapidly mixed with 20 milliliters of a thrombin-CaCl₂ solution (human thrombin, approximately 10 IU/mL and 5 mM CaCl₂) and poured into a Petri dish (ID=8.4 cm), at first allowed to stand undisturbed at room temperature for approximately 30 minutes and then incubated for approximately 16 hours in a humid chamber at 37° C. The clot in the form of a round disc having a thickness of approximately 7 millimeters was then deep-frozen and lyophilized.

The lyophilisate formed (fleece-like flat material) had a light, fine-porous appearance and substantially retained the form of a clot prior to lyophilization. The flat material was incubated for approximately 3 hours in humid chamber at room temperature, whereby a soft, adaptable and highly absorbent biopolymer product was obtained. The biopolymer product had a residual moisture content of about 15% and a water absorption capacity of approximately 3-fold of its own weight.

The biopolymer product was then wetted with water for about 15 minutes to substantially fill it with water.

The biopolymer product was then compressed between two pieces of microporous polyethylene terephthalate film to define a biopolymer membrane. The microporous film had a thickness of about 5 μm and a porosity of about 5.0 μm. The pressure exerted was about 1.5 kg/cm² for about 5 minutes, leaving less than about 10% by weight of water in the biopolymer product. The thickness of the biopolymer product was about 7 millimeters before compression and about 75 microns thereafter. The biopolymer membrane had less than about 2% water by weight and a specific gravity or density of about 1.77 g/cm³.

The biopolymer membrane was then cut into two pieces. The first piece was placed into a 2% glutaraldehyde solution for scanning electron microscope (SEM), and the second piece was rehydrated in water for one hour before being placed into a 2% glutaraldehyde solution for SEM. FIGS. 1 to 3 are cross-sectional views of the first piece of the biopolymer membrane at different magnifications, while FIGS. 4 to 6 are cross-sectional views of the second piece at different magnifications. The first (non-hydrated) piece had a thickness of about 70-75 μm, while the second piece had a thickness of about 115-120 μm. The Figures show the biopolymer membrane having a barrier-like structure having pores with a diameter of less than about 1 μm and cracks with a thickness of less than about 1 μm. The biopolymer membrane was flexible and had a curvature radius of 2 centimeters without the formation of cracks at the surface of the membrane. The membrane can be cut and sutured, as is shown in FIG. 13.

### Example 2

Example 1 was repeated in substantial form except that the gel was prepared by sequentially mixing the fibrinogen and thrombin components. FIGS. 7 to 9 are cross-sectional view of the dehydrated biopolymer membrane, and FIGS. 10 to 12 are cross-sectional views of the hydrated biopolymer membrane. The biopolymer membrane of example 2 has two opposed outer layers sandwiching a fibrin porous layer there between. The outer layers of this embodiment are more regular, dense and homogeneous that that in Example 1.

As shown in the Figures, the structure of the biopolymer membranes of Examples 1 and 2 are quite different. The biopolymer membrane of Example 1 is more compact while the biopolymer membrane of Example 2 has larger cells pores. Additionally, the residence time of the biopolymer membrane of Example 1 in a rat's body is relatively higher than the residence time of the biopolymer membrane of Example 2 in a comparable rat's body. Depending on the thickness of the membrane, the inventor found that the residence time could be varied by as much as twelve weeks.

### Example 3

The biopolymer membrane of Example 1 was lyophilized to lower the solvent (water) content to less than about 0.2% by weight.

### Example 4

The biopolymer membrane of Example 1 was repeated, except that the biopolymer product was washed by percolating water through it. After washing, the biopolymer product had a thrombin content of less than about 0.2 IU/cm³. The washed biopolymer product was then further treated as in Example 1.

### Example 5

The biopolymer product of Example 1 can be repeated except substituting photoactivable thrombin for thrombin, which can be activated for the preparation of the biopolymer product and then further treated as in Example 1.

### Example 6

The biopolymer product of Example 4 can be repeated except that after the compression step, the photoactivable thrombin is further activated.

### Example 7

Example 1 can be repeated where photoactivable thrombin is used instead of thrombin and the compression step is carried out in two successive steps. After activating the thrombin and wetting the biopolymer product, it can be compressed so that about 60% of the water by weight is removed. Any thrombin remaining in the compressed biopolymer product can then be light-activated, and the biopolymer product further compressed (as in Example 1) to define the biopolymer membrane of the invention. Of course, the membrane could be dried to further lower the solvent content.

### Example 8

The biopolymer membrane of Example 1 was dipped in an aqueous solution containing glutaraldehyde 1% by weight. After hydrating the membrane for about one hour, it was washed with sterile water and then air-dried.

### Example 9

The biopolymer membrane of Example 1 was further air-dried, so as to lower the water content to less than about 0.5% by weight.

### Example 10

The biopolymer membrane of Example 6 was dipped in an aqueous solution containing glutaraldehyde 1% by weight. After hydrating the membrane for about one hour, it was washed with sterile water and then air-dried.

### Examples 11 to 21

Example 1 can be repeated where the biopolymer product is wetted with an aqueous solution having an additive. Suitable additives are set forth in the following Table 1.

TABLE 1

| | | mg/ml of |
|---|---|---|
| Example | Additive | solution |
| 11 | Bone growth factor BMP | 1 |
| 12 | Osteoblast | 1 |
| 13 | Indomethacin | 3 |
| 14 | Paracetamol | 3 |
| 15 | Glycerol | 5 |
| 16 | A frazzled polypeptide that | |
| | inhibits or prevents a graft | |
| | rejection | |
| 17 | Calcium glycerophosphate | 2 |
| 18 | Morphine | 1 |
| 19 | Sulfamide | 2 |
| 20 | Sodium polyethylene | 3 |
| 21 | Zocor ® | 2 |

### Example 22 to 33

Example 1 was repeated except that the biopolymer product was wetted with a solution containing a polymerizable biomaterial (5% by weight). The solution chosen for each respective example is set forth in Table 2. The compression was carried out by placing the biopolymer product on a water-permeable membrane, which prevented the passage of the polymerizable biomaterial.

TABLE 2

| Example | Biomaterial added |
|---|---|
| 22 | chondroitin-4 sulfate |
| 23 | dermatan sulfate |
| 24 | keratan sulfate |
| 25 | hyaluronic acid |
| 26 | Chitosan |
| 27 | Alginate |
| 28 | Laminin |
| 29 | Fibronectin |
| 30 | Elastin |
| 31 | Collagen |
| 32 | Collagen 50% + fibronectin |
| | 50% |
| 33 | Fibronectin 50% + alginate |
| | 50% |

### Example 35

Example 1 was repeated for preparing two biopolymer membranes. The membranes were placed inside a container having two vertical walls. The solution for preparing the biopolymer product of Example 1 was then introduced into the container. After lyophilizing the gelled solution, a three-layered membrane was defined having two opposed outer layers and an intermediate layer disposed there between. Each outer layer was a biopolymer membrane of the invention, and the intermediate layer was the biopolymer product of the invention.

### Example 36

Example 1 can be repeated except that during the compression, the biopolymer product is stretched in two directions orthogonal to the compression direction.

### Example 37

Example 1 was repeated except that the wetted biopolymer product was stretched in one direction, expelling about 15% of the solvent by weight of the biopolymer product.

### Example 38

Example 1 was repeated except that the biopolymer product was compressed with variable pressures. Specifically, the biopolymer product was pressed between a first substantially flat plate and a first embossed plate having a top and a valley, so that the thickness of the biopolymer product between the top of the embossed plate and the flat plate was about 50 μm and the thickness of the biopolymer product between the valley of the embossed plate and the flat plate was about 250 μm. The pressure exerted on the portion of the biopolymer product between the top of the embossed plate and the flat plate was about 5 kg/cm².

### Examples 39 to 45

Example 1 was repeated except that different static pressures, or different pressure profiles, were used as set forth Table 3.

TABLE 3

| Example | Pressure or Pressure Profile |
|---|---|
| 39 | 0.5 kg/cm² |
| 40 | 0.250 kg/cm² |
| 41 | 0.150 kg/cm² |
| 42 | 2 kg/cm² |
| 43 | Continuously progressive increase of |
| | the pressure up to 1 kg/cm² within 5 |
| | minutes |
| 44 | Continuously progressive increase of |
| | the pressure up to 10 kg/cm² within 10 |
| | minutes |
| 45 | Continuously progressive increase of |
| | the pressure up to 10 kg/cm² within 15 |
| | minutes |
| 46 | Initial pressure of about 0.5 kg/cm² can |
| | be continuously and progressively |
| | increased to about 2 kg/cm² within 5 |
| | minutes |
| 47 | Initial pressure of about 0.5 kg/cm² can |
| | be continuously and progressively |
| | increased to about 2 kg/cm² within 10 |
| | minutes |

Naturally, any predetermined pressure or pressure profile may be employed, depending on the intended use may be determined by one skilled in the art.

### Example 48

Example 1 can be repeated for the preparation of a biopolymer product having a thickness of about 20 millimeters. The biopolymer product can then be cut such that an inox mandrel coated with Teflon® can be inserted there through. The biopolymer product can then be wetted with water (physiological buffers could also be used) and compressed between two plates such that the pressure between the mandrel and one of the plates is about 5 kg/cm². After removing the mandrel, the resulting biopolymer membrane will have a channel in the void created by the mandrel.

### Example 49

Example 48 can be repeated such that membrane has a plurality of channels.

### Example 50

Example 1 was repeated except that the biopolymer product was wetted with an aqueous solution of tricalcium phosphate (10 mg/ml).

### Examples 51 to 62

Example 35 was repeated except that the intermediate biopolymer product was prepared with a second biomaterial, which is set forth in Table 4:

TABLE 4

| EXAMPLE | Additional Biomaterial |
|---|---|
| 51 | Chondroitin-4 sulfate |
| 52 | dermatan sulfate |
| 53 | keratan sulfate |
| 54 | hyaluronic acid |
| 55 | Chitosan |
| 56 | Alginate |
| 57 | Laminin |
| 58 | Fibronectin |
| 59 | Elastin |
| 60 | Collagen |
| 61 | Collagen 50% + fibronectin |
| | 50% |
| 62 | Fibronectin 50% + alginate |
| | 50% |

### Example 63

The biopolymer membrane of Example 1 can be prepared and then wetted with water and rolled around a mandrel, so that portions of the membrane overlapped each other. The overlapped portions can then glued together with fibrin glue.

### Example 64

Example 1 can be repeated except that the biopolymer solution to be gelled is cast in a mold provided with a methylcellulose lattice, so that the lattice is located within the gel. After lyophilization, the lattice will be located within the biopolymer product. After compression, the lattice is disposed within the biopolymer membrane.

### Example 65

Example 64 can be modified such that the lattice is disposed on a face of the membrane.

### Example 66

The biopolymer membrane of Example 1 was repeated and then wetted so as to increase its flexibility. The wetted membrane was then cut into separate pieces, each of which could be stretched about 20% longer than its original length.

### Example 67

The biopolymer membrane of Example 66 was then hydrated in an aqueous solution of 1% glutaraldehyde by weight.

### Example 68

Example 1 can be repeated where the biomaterial and thrombin are polymerized in a mold having an inflatable balloon and walls defining the shape of a heart sock. The biomaterial and thrombin may be brushed, or preferably sprayed, into the mold. Upon polymerization, the balloon is inflated to compress the gel against the walls of the mold to a thickness of about 0.5 centimeters to about 0.8 centimeters. The gel layer can then be lyophilized to produce the biopolymer product of the invention on a wall of the mold. Additionally, the biopolymer product can be wetted with an aqueous solution of tannic acid (1% by weight) to prevent tissue calcification. Finally, the mold may have a film such as polytetrafluoroethylene, silicone, or aluminum to facilitate the removal of the biopolymer product. In other embodiments, the biopolymer membrane may be prepared directly on the film.

### Example 69

Example 68 was modified where the biomaterial and thrombin were simultaneously mixed and then introduced into the mold in a single application such that the gel was polymerized essentially in the mold. Once lyophilized, the biopolymer product was disposed on the upper and lower faces of the mold. The biopolymer product was then hydrated and compressed to define a biopolymer membrane of the invention. The resulting biopolymer membrane had an upper layer and a lower layer each with a porosity of less than about 1 micron, in some instances less than about 0.10 micron, and in further instances less than about 0.01 micron. The membrane also had no cracks having a diameter greater than about 1 micron.

### Example 70

A first gel can be prepared as in Example 1 but in a substantially cylindrical mold having an inner mandrel. The first gel is then lyophilized to define a biopolymer product around the mandrel. The mandrel-product combination is placed in a second substantially cylindrical mold, and a solution comprising fibrinogen and thrombin is cast in the second mold and polymerized to form a second gel around the first biopolymer product-mandrel structure. The thrombin concentration of this solution is lower than the thrombin concentration used to prepare the first gel. The second gel is then lyophilized to form a second biopolymer product on the first biopolymer product, thus defining a multilayered biopolymer product. The multilayered product is then hydrated with water (a physiological buffer may also be used). Thereafter, the wetted multilayered product is compressed at about 5 kg/cm² to form a multilayered biopolymer membrane, which can then be removed from the mandrel.

### Example 71

The biopolymer membrane of Example 1 was placed in the bottom of a 1 square centimeter cup (any size cup is possible) into which a thrombin solution was introduced. The solution was then lyophilized to produce a layer of thrombin on a face of the biopolymer membrane.

### Example 72

The biopolymer membrane of Example 1 was placed in the bottom of a 1 square centimeter cup (any size is possible) into which a fibrinogen solution was introduced. The solution was then lyophilized to produce a layer of fibrinogen on a face of the biopolymer membrane.

### Example 73

Example 72 can be repeated except that after the layer of fibrinogen is formed, the membrane is placed back into the cup, and then a thrombin solution is introduced into the cup and onto the membrane. The solution is then lyophilized to produce a layer of thrombin on the layer of fibrinogen.

### Example 74

Example 1 was repeated except that the compression of the biopolymer product was carried out between a first polyethyleneterephthalate membrane having a thickness of about 15 microns and a porosity of about 5 microns and a second polyethyleneterephthalate membrane 35 having a thickness of about 15 microns and a porosity of about 25 microns. During the compression, water was expelled through the pores of the first and second membranes.

### Example 75

Example 74 may be modified where a vacuum of a sintered glass support or a microporous metal filter is exerted on the second membrane.

### Example 76

Example 1 was repeated except that the fibrinogen was obtained from the blood of a human patient. Upon compression, an autologous biopolymer membrane was prepared. Of course, the fibrinogen from any mammal may be used.

### Example 77

Example 35 can be repeated for the preparation of a multilayered biopolymer film comprising two outer membranes with a biopolymer product disposed there between. A thrombin solution is then poured onto an outer face of the first membrane and then lyophilized to form a layer of thrombin on the outer face of the first membrane. The outer face is opposite an inner face, the inner face being in contact with the biopolymer product. A thrombin solution is then poured onto an outer face of the second membrane and then lyophilized to form a thrombin layer on the outer face of the second membrane.

### Example 78

Example 1 can be modified where the thrombin solution is mixed with a tricalcium phosphate solution before being mixed simultaneously or sequentially with fibrinogen. The concentration of the tricalcium phosphate solution is preferably from about 50 mg/ml to about 1000 mg/ml. The resulting gel can then be lyophilized to define a biopolymer product having tricalcium phosphate. The biopolymer product may then be compressed to form the biopolymer membrane of the invention.

### Example 79

It is possible to dispose the biopolymer product and/or membrane of the invention on a compatible object of choice. For instance, fibrinogen may be simultaneously or sequentially mixed with a solution of light-activable thrombin to define a bath. The light-activable thrombin may require two specific wavelengths for full activation. Next, the object of choice is introduced into the bath. Alternatively, the bath may be sprayed or brushed onto the object. The object is preferably insoluble in the bath at ambient temperature. The thrombin is then activated to define the gel of the invention on the object. The object is then removed from the bath and lyophilized, to define the biopolymer product of the invention on the object. Of course, the biopolymer product may then be hydrated compressed to define the biopolymer membrane of the invention on the object.

### Example 80

In another embodiment, the biopolymer membrane of Example 1 was hydrated and then cut to predetermined dimensions to begin a graft construction process. The cut membrane was then wrapped onto a mandrel having a preloaded inner stent. An outer stent was then applied to the wrapped membrane to a side opposite in contact with the mandrel. The outer stent may also be transferred to the outside of the membrane (such as in a helical pattern) as described by the Rapidgraft™ process of Ramus Medical Technologies. Alternatively, the biopolymer product of the invention may be wrapped onto the support and then compressed, forming a coating of the biopolymer membrane of the invention on the support.

It will be understood that the invention may be embodied in other specific forms without departing from the spirit or central characteristics thereof. The present examples and embodiments, therefore, are to be considered in all respects as illustrative and not restrictive, and the invention is not to be limited to the details given herein.

What I claim is:

1. A multilayered biocompatible structure comprising:
a biopolymer membrane; and
a biopolymer product in contact with the biopolymer membrane; wherein the biopolymer membrane in its substantially dry form has a thickness equal to or less than about 75 microns, a solvent content less than about 5% by weight of the membrane, a radius of curvature of less than about 5 centimeters, a density greater than about 1 g/cm³, and a maximum pore size of about 20 microns.

2. The structure of claim 1 wherein the biopolymer membrane comprises a blend of a biomaterial and thrombin.

3. The structure of claim 2 wherein the biomaterial is autologous.

4. The structure of claim 3 wherein the biomaterial is selected from the group consisting of fibrin, fibrinogen, chondroitin-4 sulfate, dermatan sulfate, keratan sulfate, hyaluronic acid, chitosan, chitin, alginate, laminin, elastin, fibronectin, collagen, proteoglycan, glycosaminoglycan, and mixtures thereof.

5. The structure of claim 1 wherein the biopolymer product comprises a blend of a biomaterial and thrombin.

6. The structure of claim 5 wherein the biomaterial is autologous.

7. The structure of claim 6 wherein the biomaterial is selected from the group consisting of fibrin, fibrinogen, chondroitin-4 sulfate, dermatan sulfate, keratan sulfate, hyaluronic acid, chitosan, chitin, alginate, laminin, elastin, fibronectin, collagen, proteoglycan, glycosaminoglycan, and mixtures thereof.

8. The structure of claim 1 wherein the structure further comprises an additive mixed with the biopolymer membrane or the biopolymer product.

9. The structure of claim 8 wherein the additive is selected from the group consisting of processing aids, a radioactive marker, a calcium containing compound, an antibody, an antimicrobial agent, an agent for improving the biocompatibility of the structure, proteins, an anticoagulant, an anti-inflammatory compound, a compound reducing graft rejection, any living cell, cell growth inhibitors, agents stimulating endothelial cells, antibiotics, antiseptics, analgesics, antineoplastics, polypeptides, protease inhibitors, vitamins, cytokine, cytotoxins, minerals, proteins, interferons, hormones, polysaccharides, genetic materials, proteins promoting or stimulating the growth and/or attachment of endothelial cells on the cross-linked biopolymer, growth factors, cell growth factors, growth factors for heparin bond, tannic acid, nerve growth factor, neurotrophic factor (NTFs), neurothrophin 3 (NT3), brain derived NTF (BDNTF), cilary NTF (CNTF), substances against cholesterol, pain killers, collagen, osteoblasts, chondroblasts, chondrocytes, osteoclasts, hematpoeitic cells, stromal cells, osteoprogenitor cells, keratinocytes cells, anti coagulants, poly DL lactate, alginate, recombinant material, triglycerides, fatty acids, C₁₂-C₂₄ fatty acids, collagen, any pharmaceutical agent, activable factor VII, activable factor IX, activable factor X, activable factor XI, activable plasmin, photoactivable t-PA, photoactivable urokinase, taxol, cytostatic agent, antigenic agent, plasminogen, compounds activating the conversion of plasminogen into plasmin, compounds inhibiting the conversion of plasminogen in plasmin, and mixtures thereof.

10. The structure of claim 9 wherein the processing aid is a cryoprotectant.

11. The structure of claim 10 wherein the cryoprotectant is glyercol, dimethyl sulfoxide, or trehalose.

12. The structure of claim 9 wherein the radioactive marker is Technitium-99-m-HDP or an iodine isotope.

13. The structure of claim 9 wherein the substances against cholesterol are statins or stanols.

14. The structure of claim 9 wherein the pharmaceutical agent is selected from the group consisting of antibiotics, antiseptics, analgesics, and antineoplastics.

15. The structure of claim 9 wherein the compound that activates the conversion of plasminogen into plasmin is selected from the group consisting of t-PA, u-KA, su-PA, and streptokinase.

16. The structure of claim 9 wherein the compound that inhibits the conversion of plasminogen in plasmin is selected from the group consisting of aprotinin, tranexanic acid, a2-antiplasmins, a2-macroglobulins, a2-antitrypsin, antithrombin, antistreptokinase, aminocapronic acid, tranexamic acid, C1-esterase inhibitor, and anti-urokinase.

17. The structure of claim 1 wherein the biopolymer membrane is cross-linked.

18. The structure of claim 1 wherein the biopolymer membrane is sterilized.

19. The structure of claim 2 wherein the thrombin is natural, recombinant, or a mixture thereof.

20. The structure of claim 5 wherein the thrombin is natural, recombinant, or a mixture thereof.

21. The structure of claim 2 wherein the thrombin is activable.

22. The structure of claim 5 wherein the thrombin is activable.

23. The structure of claim 1 wherein the thickness of the biopolymer membrane is equal to or less than about 45 microns.

24. The structure of claim 1 wherein the maximum pore size of biopolymer membrane is about 10 microns.

25. The structure of claim 1 wherein the maximum pore size of biopolymer membrane is about 5 microns.

26. The structure of claim 1 wherein the maximum pore size of biopolymer membrane is about 1 micron.

27. The structure of claim 1 wherein the maximum pore size of biopolymer membrane is about 0.10 micron.

28. The structure of claim 1 wherein the maximum pore size of biopolymer membrane is about 0.01 micron.

29. A multilayered biocompatible structure comprising:
a first blend of a biomaterial and thrombin defining a biopolymer membrane;
a second blend of a biomaterial and thrombin defining a biopolymer product;
wherein the biopolymer membrane contacts the biopolymer product; and
wherein the biopolymer membrane in its substantially dry form has a thickness equal to or less than about 75 microns, a solvent content less than about 5% by weight of the membrane, a radius of curvature of less than about 5 centimeters, a density greater than about 1 g/cm³, and a maximum pore size of about 20 microns.

30. The multilayered biocompatible structure of claim 29 wherein the biomaterial of the first blend is fibrinogen.

31. The multilayered biocompatible structure of claim 29 wherein the thickness of the biopolymer membrane is equal to or less than about 45 microns.

32. A biopolymer membrane comprising:
a blend of a biomaterial and thrombin, characterized in that the membrane in its substantially dry form has a thickness equal to or less than about 75 microns, a solvent content less than about 5% by weight of the membrane, a radius of curvature of less than about 5 centimeters, a density greater than about 1 g/cm³, and a maximum pore size of about 20 microns.

33. The biopolymer membrane of claim 32 wherein the biomaterial is fibrinogen.

34. The biopolymer membrane of claim 32 wherein the thickness is equal to or less than about 45 microns.

35. The biopolymer membrane of claim 32 wherein the maximum pore size is about 10 microns.

36. The biopolymer membrane of claim 32 wherein the maximum pore size is about 1 micron.

37. The biopolymer membrane of claim 34 wherein the maximum pore size is about 1 micron.

38. A multilayered biopolymer membrane comprising:
a first biopolymer membrane having has a thickness equal to or less than about 75 microns, a solvent content less than about 5% by weight of the membrane, a radius of curvature of less than about 5 centimeters, a density greater than about 1 g/cm³, and a maximum pore size of about 20 microns; and
a second biopolymer membrane having a thickness equal to or less than about 45 microns, a solvent content less than about 5% by weight of the membrane, a radius of curvature of less than about 5 centimeters, a density greater than about 1 g/cm³, and a maximum pore size of about 5 microns; and
wherein the first biopolymer membrane contacts the second biopolymer membrane.

39. An artificial skin comprising:
a blend of thrombin and fibrinogen defining a biopolymer membrane in its substantially dry form having a thickness equal to or less than about 75 microns, a solvent content less than about 5% by weight of the membrane, a radius of curvature of less than about 5 centimeters, a density greater than about 1 g/cm³, and a maximum pore size of about 20 microns;
a first set of cells selected from the group consisting of fibroblast cells, endothelial cells, and a mixture thereof, wherein the first set of cells contact the biopolymer membrane; and
a second set of cells selected from the group consisting of epithelial cells, keratinocyte cells, and a mixture thereof, wherein the second set of cells contact the biopolymer membrane.

40. The artificial skin of claim 39 wherein the thickness of the biopolymer membrane is equal to or less than about 45 microns.

41. A process for forming a biopolymer membrane comprising:
mixing a biomaterial and thrombin in a solvent to define a gel;
drying the gel to define a sponge having a solvent content;
adjusting the solvent content of the sponge so that the sponge is substantially filled with the solvent; and
compressing the sponge to define a biopolymer membrane in its substantially dry form having a thickness less than about 75 microns and a solvent content less than about 5% by weight of the membrane, characterized in that the membrane has a radius of curvature of less than about 5 centimeters, a density greater than about 1 g/cm³, and a maximum pore size of about 20 microns.

42. The process of claim 41 wherein the biomaterial is autologous.

43. The process of claim 41 wherein the biomaterial is selected from the group consisting of fibrin, fibrinogen, chondroitin-4 sulfate, dermatan sulfate, keratan sulfate, hyaluronic acid, chitosan, chitin, alginate, laminin, elastin, fibronectin, collagen, proteoglycan, glycosaminoglycan, and mixtures thereof.

44. The process of claim 41 wherein the mixing is simultaneous or sequential.

45. The process of claim 41 wherein the compressing comprises at least two compressions.

46. The process of claim 41 wherein the drying is carried out by lyophilization, osmosis, centrifugation, compression, or a mixture thereof.

47. The process of claim 41 further comprising washing the biopolymer membrane.

48. The process of claim 41 wherein the thrombin is natural, recombinant, or a mixture thereof.

49. The process of claim 41 wherein the thrombin is activable.

50. The process of claim 49 further comprising activating the thrombin.

51. The process of claim 50 wherein the activating is carried out by photoactivation or radiation.

52. The process of claim 41 further comprising adding an additive selected from the group consisting of processing aids, a radioactive marker, a calcium containing compound, an antibody, an antimicrobial agent, an agent for improving the biocompatibility of the structure, proteins, an anticoagulant, an anti-inflammatory compound, a compound reducing graft rejection, any living cell, cell growth inhibitors, agents stimulating endothelial cells, antibiotics, antiseptics, analgesics, antineoplastics, polypeptides, protease inhibitors, vitamins, cytokine, cytotoxins, minerals, proteins, interferons, hormones, polysaccharides, genetic materials, proteins promoting or stimulating the growth and/or attachment of endothelial cells on the cross-linked biopolymer, growth factors, cell growth factors, growth factors for heparin bond, tannic acid, nerve growth factor, neurotrophic factor (NTFs), neurothrophin 3 (NT3), brain derived NTF (BDNTF), cilary NTF (CNTF), substances against cholesterol, pain killers, collagen, osteoblasts, chondroblasts, chondrocytes, osteoclasts, hematpoeitic cells, stromal cells, osteoprogenitor cells, keratinocytes cells, anti coagulants, poly DL lactate, alginate, recombinant material, triglycerides, fatty acids, C₁₂-C₂₄ fatty acids, collagen, any pharmaceutical agent, activable factor VII, activable factor IX, activable factor X, activable factor XI, activable plasmin, photoactivable t-PA, photoactivable urokinase, taxol, cytostatic agent, antigenic agent, plasminogen, compounds activating the conversion of plasminogen into plasmin, compounds inhibiting the conversion of plasminogen in plasmin, and mixtures thereof.

53. The process of claim 41 further comprising sterilizing the biopolymer membrane.

54. The process of claim 53 wherein the sterilizing agent is a physical agent or a chemical agent.

55. The process of claim 54 wherein the physical agent is selected from the group consisting of heat, radio frequency, gamma radiation, ion-beam, and electron beam radiation.

56. The process of claim 54 wherein the chemical agent is ethylene oxide.

57. The process of claim 41 further comprising drying the membrane.

58. The process of claim 41 further comprising cross-linking the biopolymer membrane.

59. The process of claim 58 wherein the cross-linking is effectuated with a cross-linking agent selected from the group consisting of aldehydes, diimides, enzymes, tri-hydroxybenzene carboxylic acids, and mixtures thereof.

60. The process of claim 59 wherein the tri-hydroxybenzene carboxylic acid is tannic acid.

61. The process of claim 59 wherein the aldehyde is formaldehyde or glutaraldehyde.

62. The process of claim 59 wherein the enzyme is factor XIII.

63. The process of claim 41 wherein the solvent is aqueous, organic, or a mixture thereof.

64. The process of claim 63 wherein the organic solvent is selected from the group consisting of cremophor, polyethyleneglycol, polysorbate.

65. The process of claim 41 further comprising stretching the biopolymer membrane.

66. The process of claim 41 further comprising associating the biopolymer membrane to a lattice.

67. A process for forming a multilayer biopolymer membrane comprising:
providing a first biopolymer membrane in its substantially dry form having a thickness less than about 75 microns, a solvent content less than about 5% by weight of the membrane, a radius of curvature of less than about 5 centimeters, a density greater than about 1 g/cm³, and a maximum pore size of about 20 microns;
providing a second biopolymer membrane in its substantially dry form having a thickness less than about 75 microns, a solvent content less than about 5% by weight of the membrane, a radius of curvature of less than about 5 centimeters, a density greater than about 1 g/cm³, and a maximum pore size of about 20 microns; and
contacting the first biopolymer membrane to the second biopolymer membrane to define a multilayer biopolymer membrane.

68. The process of claim 67 wherein the first and second biopolymer membranes are of a different composition.

69. The process of claim 67 wherein the first and second biopolymer membranes each comprise a biomaterial selected from the group consisting of fibrin, fibrinogen, chondroitin-4 sulfate, dermatan sulfate, keratan sulfate, hyaluronic acid, chitosan, chitin, alginate, laminin, elastin, fibronectin, collagen, proteoglycan, glycosaminoglycan, albumin, globulins, and mixtures thereof.

70. The process of claim 67 wherein the first and second biopolymer membranes each have a thickness that is different from the other.

71. The process of claim 67 wherein the thickness of the first biopolymer membrane is equal to or less than about 45 microns.

72. The process of claim 67 wherein the thickness of the second biopolymer membrane is equal to or less than about 45 microns.
